(19) United States
(12) Patent Application Publication
(10) Pub. No.: US 2024/0225616 A9
(43) Pub. Date: Jul. 11, 2024

(54) APPARATUS FOR MANUAL ACQUISITION OF A BIOLOGICAL SAMPLE AND USE THEREOF

(71) Applicants: Peter James Webber, Berriew Welshpool (GB); XX02 CELL LTD, London (GB)

(72) Inventor: Peter James Webber, Berriew Welshpool (GB)

(21) Appl. No.: 18/492,747

(22) Filed: Oct. 23, 2023

(30) Foreign Application Priority Data
Oct. 24, 2022 (GB) 2215754.9

(51) Int. Cl. A61B 10/00

(52) U.S. Cl. CPC A61B 10/0045; A61B 2010/0074

(57) ABSTRACT

A self-sampling apparatus for collection of a cellular sample, particularly a vaginal sample for detection of human papillomavirus and its use is disclosed. The apparatus has an evacuated sample container closed by a puncturable seal and a fluid sample chamber in fluid communication with the evacuated container via a needle assembly. A fluid sample is drawn into the evacuated sample container upon establishing fluid communication between the fluid sample chamber and the evacuated container.

### CROSS-REFERENCE TO RELATED APPLICATIONS

This is a U.S. Nonprovisional Application that claims priority to Great Britain Application No. 2215754.9 filed on Oct. 24, 2022, which is hereby incorporated by reference herein in its entirety.

### FIELD OF THE INVENTION

This invention relates to an apparatus for manual acquisition of cellular material samples, use thereof of an evacuated container for collection of cellular material for analysis and to a method of manually acquiring a vaginal sample for analysis. The invention relates particularly to an apparatus for vaginal self-sampling of a liquid biological sample for screening for biological indicators, for example, human papillomavirus (HPV).

### BACKGROUND

A wide range of medical conditions may be detected by analysis of biological samples taken from the body for analysis for agents implicated in causing the condition, for example cervical cancer. Samples may be gathered in a range of ways including by surgical removal and obtaining cellular material using swabs, spatulas, brushes or other implements. However, samples may be prone to drying out unless appropriately stored prior to analysis.

Many medical conditions may be preventable and have an improved chance of successful treatment if the condition or indicators for it are detected early enough. However, screening programs typically rely on people attending health clinics to provide samples for laboratory testing and uptake of screening may often be poor due to the logistical or cultural barriers or for personal reasons. Self-sampling may help to address such difficulties. However, obtaining an appropriate sample and it remaining in a suitable form for reliable analysis may be problematic.

### SUMMARY

It has been found that fluid specimens or samples may be obtained and reliably stored pending analysis by self-sampling by the subject using an evacuated container in fluid communication with a fluid sample taken directly from a chamber in which the sample has been collected or directed from the body.

In a first aspect, the invention provides a method of manually acquiring a cellular fluid sample, preferably a vaginal sample, comprising providing an evacuated sample container having a puncturable seal and a needle assembly adapted to receive a vaginal sample and piercing the puncturable seal to receive the cellular fluid sample into the evacuated container.

The needle assembly suitably receives a fluid sample from a chamber in which the sample is collected or directed from the body of a subject, for example from a sample collector or probe employed to directly acquire the cellular sample from the subject. The sample may be in fluid form or in solid form in which case, the solid sample is combined with a transport medium to provide the sample as a fluid comprising the solid sample dispersed or dissolved in the transport medium. The transport medium may also provide a chemical or biological function such as a storage medium or may comprise components to provide such a function to preserve the cellular sample. In a preferred embodiment, the transport medium comprises SUREPATH™ preservative, available from CellPath or preservation solution known in the field may be employed, for example PreservCyt solution available from Hologic.

In a second aspect, the invention provides a self-sampling apparatus for collection of a cellular sample preferably a vaginal sample comprising an evacuated sample container having an openable end which is closed by a puncturable seal and a fluid sample chamber adapted to be, in use, in fluid communication with the evacuated container through the puncturable seal such that a fluid sample in the fluid sample chamber is drawn into the evacuated sample container upon establishing fluid communication between the fluid sample chamber and the evacuated container.

The puncturable seal may take any suitable form capable of being punctured by a needle including a septum and a puncturable stopper.

Suitably, the evacuated container and the fluid sample chamber are, in use, connected by fluid communication means, for example, a needle such that upon piercing the puncturable seal, the needle provides a conduit between the fluid sample chamber and the evacuated container and a pressure gradient between the chamber and the container is established which draws the fluid sample from the fluid sample chamber into the evacuated chamber. There is a pressure gradient across the fluid communication means.

In a preferred embodiment, the fluid communication means comprises a needle, especially a needle with a protective shroud. In one embodiment, the needle is connectable to or integral with the fluid sample chamber, for example the needle may be part of a needle assembly comprising a needle holder adapted to be connected to an outlet of the fluid sample chamber and the needle. Alternatively, the needle assembly may be connected to or integral with the fluid sample chamber.

The needle assembly preferably further comprises a shroud for the needle for safety. The shroud is suitably dimensioned to fit over the open end of the evacuated container.

Fluid communication between the fluid sample chamber and the evacuated chamber is established by positioning the needle on the puncturable seal and, if present, the needle shroud engages or at least surrounds the open end of the evacuated container and, upon urging the needle through the puncturable seal, the fluid sample is drawn into the evacuated container.

In another embodiment, the fluid communication means comprises a double ended needle mounted in a manifold such that one needle is adapted to puncture the puncturable seal and the other end is adapted to receive a fluid cellular sample, for example from the human body or by communicating with a fluid sample chamber to establish fluid communication between the chamber and the container. Suitably, at least one and preferably both the needles are surrounded by a shroud for safety purposes. Suitably the shroud is configured and sized to engage with one or preferably both the open end of the evacuated container and an outlet in the fluid sample chamber.

A double ended needle may be used to obtain cellular samples from the body, for example, for fine needle breast biopsies, bladder cytology and the like.

The needle shroud, where present, suitably manually inter-engages with the evacuated container and/or the fluid sample chamber, for example in a push fit manner.

Suitably, the evacuated container is sterile and, upon receiving the fluid sample, is detachable from the fluid sample chamber and seals itself or may be sealed by the user. The evacuated container may be any known form of evacuated container such as evacuated containers available under the brand VACUTAINER™ from Becton Dickinson & Co. Suitably, the evacuated container self-seals upon removal of the fluid communication means. The self-sealed container containing the cellular sample may then be dispatched to a remote location for analysis.

Preferably the evacuated container, for example a standard size VACUTANER container, comprises from 5 to 25 ml, preferably 8 to 15 ml, for example 10 ml of a liquid preservative, for example SUREPATH preservative.

The fluid sample chamber may take any suitable form and includes an outlet adapted for fluid communication directly or indirectly with the evacuated container. The chamber further comprises an inlet for insertion of the cellular sample or a body carrying or containing the cellular sample taken from the body of the user. The cellular sample may be carried by a foreign body which has been inserted into a bodily orifice, for example the vagina, penis, anus, ears, nasal cavity and mouth or in an open wound or in the eye. The foreign body gains cellular material from the locality and acts as a carrier of the cellular material and is placed in the fluid sample chamber. The cellular material may then be removed or extracted from the body, for example using a transport medium or by physical action to extract cellular material from the foreign body.

Suitably the fluid sample is of sufficient volume to be readily drawn into the evacuated container upon establishing the fluid communication therebetween. As desired, the fluid sample container may contain a transport medium or a preservative or other functional fluid to solubilize, dissolve, or disperse the cellular sample and to provide sufficient fluid volume such that the fluid sample covers the outlet whereby it is drawn into the evacuated container.

The present invention is particularly suitable for use in self-sampling for human papillomavirus (HPV) which is implicated in the development of cervical cancer.

Cervical cancer is preventable and treatable when diagnosed early enough and HPV is known as a primary cause of cancer of the cervix. Molecular testing for HPV in cervical screening programs provides significant benefit to public health. The World Health Organization has recommended the use of vaginal self-sampling for the early detection of cancer of the cervix which enables logistical, personal and cultural reasons to be overcome or mitigated to some extent. A number of HPV tests are known for use in screening programs including Abbott RealTime High Risk HPV test, BD Onclarity HPV test, Cepheid Xpert JPV test, Hologic Aptima HPV test, Qiagen Hybrid Capture 2 High risk HPV DNA test and Roche Cobas 4800, 6800 and 8800 HPV systems.

Known self-sampling techniques involve the use of swabs and brushes to gather cells. NHS England has trialed swab smear kits for home testing and many other similar kits are available.

The menstrual cycle is associated with the passage of exfoliated cervical cells into the vagina. Sanitary products, for example tampons, absorb or carry such vaginal cellular material and provides a rich source of cellular material for screening for HPV. The days of menstruation and those spanning mid-cycle ovulation provide abundant cells for testing and screening processes are known where sanitary products are packaged and dispatched to laboratories for analysis. The invention provides a screening process for those at risk of cervical cancer without the barriers that some may find difficult. There remains a need to provide a simple, easy to use home or self-sampling test to encourage increased uptake and early diagnosis.

In a third aspect, the invention provides a self-sampling apparatus for collection of a vaginal cellular sample comprising an evacuated sample container having an openable end which is closed by a puncturable seal and a fluid sample chamber adapted to receive a vaginal cellular sample which, in use, is in fluid communication with the evacuated container through the puncturable seal such that a vaginal cellular fluid sample is drawn into the evacuated sample container upon establishing fluid communication between the fluid sample chamber and the evacuated container.

In a fourth aspect, the invention provides use of an evacuated sample container having a puncturable seal in combination with a needle assembly adapted to pierce the puncturable seal and a fluid sample chamber wherein the needle assembly provides a fluid communication conduit between the fluid sample chamber and the evacuated chamber wherein upon piercing the puncturable seal, whereby the vaginal cellular sample is urged from the fluid sample chamber to the evacuated container.

In a preferred embodiment, the fluid sample chamber preferably comprises a syringe or a “Moon Cup”, known as a MCUK in the USA for receiving the vaginal sample. The syringe suitably comprises a body having a syringe outlet, which is in fluid communication with the evacuated sample container, and a syringe inlet into which is placed the cellular sample or body containing the cellular sample, for example a tampon. The syringe inlet in the body is adapted to receive a syringe plunger which may be compressed to extract the cellular material from the body carrying the sample. The fluid sample chamber and the evacuated container are suitably in fluid communication through a needle assembly which is in fluid communication with the outlet of the syringe and comprises a needle in a shroud. A sanitary product carrying the vaginal sample is suitably placed in a syringe. The syringe plunger may then be depressed to expel the vaginal sample from the sanitary product into the end of the syringe. The needle may be inserted into the puncturable seal prior or after depression of the syringe plunger where upon the vaginal cellular sample removed from the sanitary product passes through the needle into the evacuated container.

In a further aspect, the invention provides a self-sampling or home-testing kit for use by a subject to acquire a sample from themselves comprising a syringe comprising a body and a plunger, the plunger being removable to present an inlet for receiving a sanitary product having a vaginal cellular sample and an outlet at the opposite end of the syringe, preferably a disposable syringe, especially a 5 ml-20 ml syringe, a needle holder, preferably having a safety shroud, adapted to attach to the syringe outlet and an evacuated container having a puncturable seal and containing a cell preservation solution the kit further comprising one or more sanitary products sized to fit in the syringe.

Preferably, the syringe and needle assembly are attached and the needle has a safety shroud.

The preservation solution is suitably present at a volume of 5 to 20 ml, especially 8 to 15 ml, for example 10 ml. Any preservation solution known in the field may be employed, for example PreservCyt solution.

Suitably, the tampon in the kit comprises a regular-sized sanitary product, for example a tampon, with an absorbency of 3 to 10 ml, preferably 5 ml, which collects vaginal cellular material during menstruation. Once inserted, the tampon captures cervical cells that have exfoliated and drifted into the vaginal fluid. The subject is familiar with the procedure and the sanitary product typically will yield large numbers of cervical cells. The used sanitary product is suitably placed string side up in the syringe. The syringe and safety needle are then attached to the top of the evacuated container and light pressure is applied to the plunger to pierce the puncturable seal and compress the sanitary product to release the vaginal cellular sample. The cellar fluid sample passes through the needle into the evacuated container containing the preservation solution. The syringe and safety needle may then be detached from the evacuated container for safe disposal.

Suitably, the sample has been obtained from the body by the user and placed in a fluid sample chamber. The sample may be obtained using a sample collector which physically acquires the cellular sample by insertion into the requisite bodily orifice. The sample collector may comprise a probe which collects cellular material from the body which is in communication with the needle assembly. The probe may be any suitable shape or construction depending on the locality from which the sample is to be taken to enable collection of sufficient cellular material to enable analysis to take place. The probe may be connected to the needle assembly directly or via a flexible conduit.

The sample collector may be in fluid communication with a syringe such that the sample may be physically located through contact or be drawn onto the collector by withdrawing the plunger of the syringe so as to provide a cellular sample on the sample collector or probe. Preferably the cellular sample passes into the syringe, for example as shown in FIG. 5 below. The syringe acts as a fluid sample chamber which is then suitably detached from the sample collector and a needle assembly may hen be attached to the syringe and the needle assembly then engaged with an evacuated container and the cellular sample conveyed into the evacuation container by depressing the syringe and/or the pressure gradient between the syringe and the evacuated container as the puncturable seal is broken by the needle.

### BRIEF DESCRIPTION OF THE DRAWINGS

The present invention is further described by way of example only with reference to the accompanying drawings, in which:

FIGS. 1A to 1C show a side elevation plan view of apparatus according to the invention at various stages in the sample acquisition process;

FIG. 2 shows a side elevation of an alternative embodiment of the invention to that shown in FIG. 1;

FIG. 3 shows a side elevation of an embodiment of the invention in FIG. 1;

FIG. 4 perspective view of another embodiment of the invention;

FIG. 5 shows a perspective view of a part of the apparatus of the invention configured for collecting a sample; and

FIGS. 6A to 6D show perspective views of other embodiments of the invention with an evacuated container connected to a sample collector.

### DETAILED DESCRIPTION

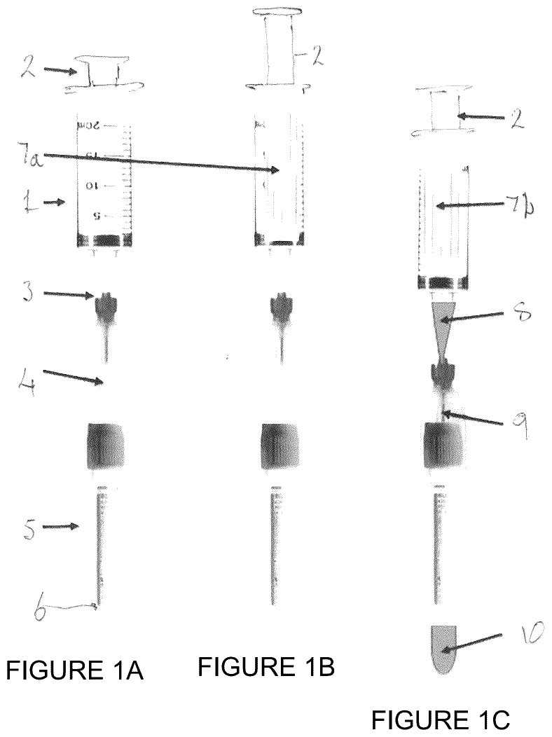

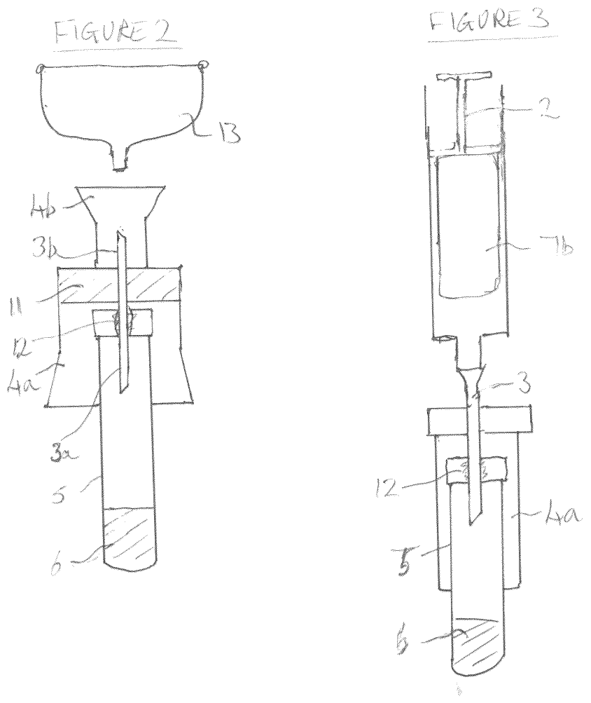

FIGS. 1A to 1C show apparatus according to the invention showing a fluid sample collection chamber 1, in this case a syringe which has a body and a plunger 2. The chamber has an outlet connected to a needle assembly 3 which has a shroud 4 for protection. The shroud 4 is dimensioned such that it fits snugly over the end of the evacuated container 5 which contain a transport fluid or preservative 6. The puncturable seal which closes the open end of the evacuated container 5 is not shown.

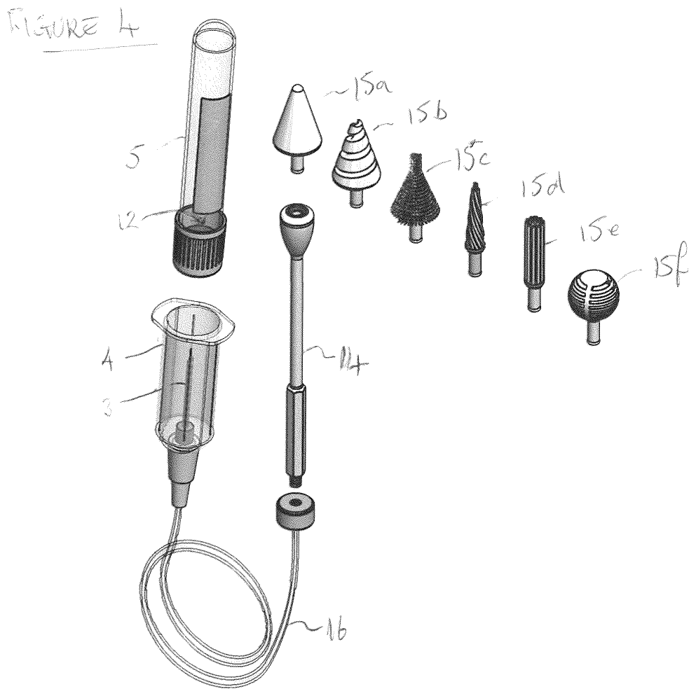

The user places a used sanitary product 7a, such as a tampon, carrying vaginal cellular material into the syringe as shown in FIG. 1B. The plunger 2 is extended to accommodate the sanitary product 7a. FIG. 3 shows the apparatus being pushed onto the top of the evacuated container 5 such that the shroud 4 snugly engages or clips onto the top of the container and sheaths the top of the container and the puncturable seal and the needle 9. As the apparatus is pressed onto the evacuated container the needle 9 punctures the puncturable seal which, the plunger 2 being depressed such that it compresses the sanitary product and expels or otherwise removes or releases cellular material from the compressed sanitary product 7b. The expelled sample passes through the outlet 8 of the syringe, through the needle 9 and into the evacuated container 5 and the preservative 10.

The needle assembly 3 and the syringe may then be removed, the puncturable seal closes and the sample is retained in the container 5 which is closed by the puncturable, fluid tight sheath.

FIG. 2 shows an apparatus according to the invention having an evacuated container 5 which contains a preservative or other fluid material. A double ended needle having a first end 3a which passes through puncturable seal or septum 12 in the open end of the container 5 and a second end 3b which is engagable with a fluid sample chamber 13 such as a Moon Cup containing the cellular sample provides a conduit for fluid communication between the container 5 and the chamber 13. The double ended needle arrangement may be employed to obtain cytological samples directly from the human or animal body. The double ended needle 31, 3b is mounted in a manifold 11 and has shrouds 4a and 4b to shield the needles 3a and 3b.

FIG. 3 shows apparatus according to the invention as shown in FIG. 1 with a tampon 7b in a compressed state and the plunger 2 depressed into the syringe, thereby to extract or expel cellular material from the tampon for exit through outlet of the syringe into needle assembly 3 and into the evacuated container 5.

FIG. 4 shows another embodiment of the invention with evacuated container 5 with septum 12 which is engagable with shroud 4 which protects against needle 3, needle 3 is in fluid communication with a fluid sample chamber 14, which acts as a sample collector, via conduit 16. The sample collector is equipped with a probe 15a to 15f depending on the particular type of sample being taken. The fluid sample chamber has an outlet which is connectable to conduit 16.

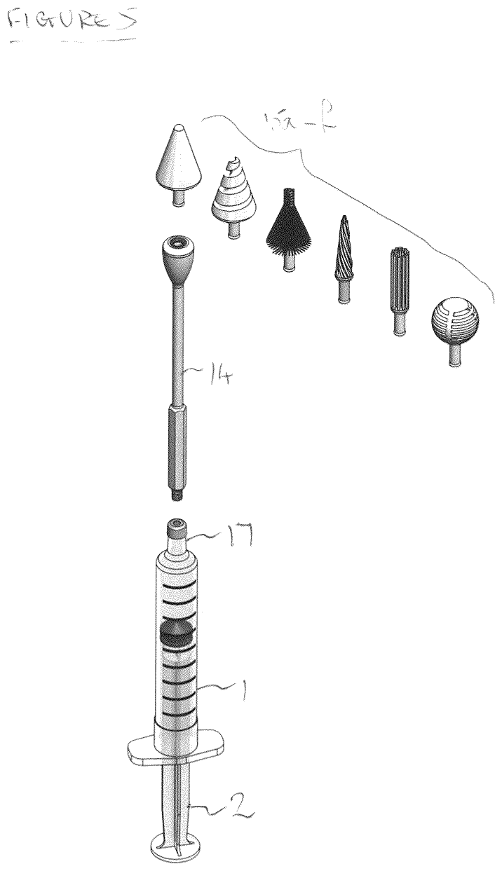

FIG. 5 shows a fluid sample chamber/sample collector with an array of optional probes 15a to 15f as shown in FIG. 4. The outlet of sample collector 14 engages with a syringe 1 via syringe outlet 17. Upon withdrawal of the plunger 2, cellular material is drawn onto the probe 15a to 15f and into the syringe vias sample collector 14. The syringe may then be disconnected from the sample collector 14 and have a needle assembly attached to the end 17 of the syringe. The syringe, containing the cellular material is then suitably connected to an evacuated container and is, in effect a fluid sample chamber. Upon depression of the plunger 2, the cellular material is expelled through outlet 17 through the needle assembly into an evacuated container to provide a cellular sample for despatch for analysis.

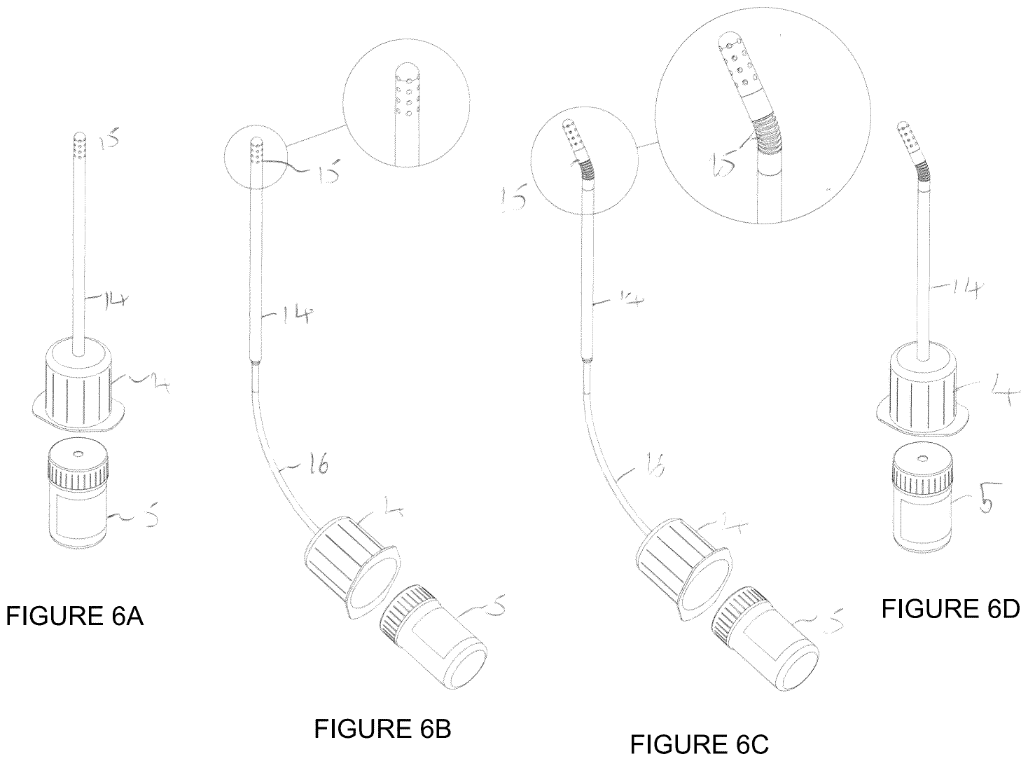

FIG. 6 shows another embodiment of the invention with evacuated container 5 and needle shroud 4 ready to be connected. The needle (not shown) within the shroud punctures the septum in the top of container 5 and the shroud and container are suitably dimensioned so as to be engaged in a push-fit manner. Sample collector 14 is in fluid communication with the container 5 in use and the probe 15 is shaped and configured to obtain cellular samples from the human or animal body. The collector and container 5 may be connected by a fluid conduit 16 which may be flexible as shown in FIGS. 6B and 6C. The probe may be flexibly mounted on the sample connector as shown in FIGS. 15C and 15D.

1. (canceled)

2. (canceled)

3. A self-sampling apparatus for collection of a vaginal cellular sample comprising an evacuated sample container having an openable end which is closed by a puncturable seal and a fluid sample chamber adapted to receive a vaginal cellular sample which, in use, is in fluid communication with the evacuated container through the puncturable seal such that a vaginal cellular fluid sample is drawn into the evacuated sample container upon establishing fluid communication between the fluid sample chamber and the evacuated container.

4. A method of manually acquiring a cellular fluid sample, preferably a vaginal sample, comprising providing an evacuated sample container having a puncturable seal and a needle assembly and a fluid sample chamber for receiving a vaginal sample wherein the needle assembly provides a fluid communication conduit between the fluid sample chamber and the evacuated chamber wherein upon piercing the puncturable seal, whereby the vaginal cellular sample is urged from the fluid sample chamber to the evacuated container.

5. A self-sampling or home-testing kit for use by a subject to acquire a sample from themselves comprising a syringe comprising a body and a plunger, the plunger being removable to present an inlet for receiving a sanitary product having a vaginal cellular sample and an outlet at the opposite end of the syringe, especially a 5 ml-20 ml syringe, a needle assembly, preferably having a safety shroud, adapted to attach to the syringe outlet, and an evacuated container having a puncturable seal and containing a cell preservation solution the kit further comprising one or more sanitary products sized to fit in the syringe wherein the needle assembly provides a fluid communication path between the syringe and the evacuated chamber.
